(19) United States
(12) Patent Application Publication
(10) Pub. No.: US 2023/0072606 A1
(43) Pub. Date: Mar. 9, 2023

(54) COMPOSITIONS AND METHODS FOR STABILIZING BIOMOLECULES

(71) Applicant: Massachusetts Institute of Technology, Cambridge, MA (US)

(72) Inventors: Robert S. Langer, Newton, MA (US); Ana Jaklenec, Lexington, MA (US); Joseph Collins, Boston, MA (US); Morteza Sarmadi, Cambridge, MA (US); Aurelien vander Straeten, Cambridge, MA (US); Maria Kanelli, West Roxbury, MA (US); John Daristotle, Bel Air, MD (US)

(21) Appl. No.: 17/903,586

(22) Filed: Sep. 6, 2022

Related U.S. Application Data
(60) Provisional application No. 63/241,317, filed on Sep. 7, 2021.

(51) Int. Cl. A61K 9/127; A61K 9/51; A61K 31/7105; A61K 47/26; A61P 37/04; A61K 39/00

(52) U.S. Cl. CPC A61K 9/1272; A61K 9/5123; A61K 31/7105; A61K 47/26; A61P 37/04; A61K 39/00; A61K 2039/53

(57) ABSTRACT

Disclosed are compositions that stabilize (e.g., thermo-stabilize) biological agents, such as mRNA. Also disclosed are methods of administering and using said compositions.

### RELATED APPLICATIONS

This application claims the benefit of priority to U.S. Provisional Patent Application No. 63/241,317, filed on Sep. 7, 2021.

### STATEMENT OF GOVERNMENT SUPPORT

This invention was made with Government support under Grant No. 75A50119C00076 awarded by the US Department of Health and Human Services (HHS). The Government has certain rights in the invention.

### BACKGROUND

Disease outbreaks, such as the ongoing COVID pandemic, have dramatically challenged global healthcare systems with huge human and financial losses. Indeed, since the start of the pandemic, more than 3 million lives have been lost and global GDP contracted by approximately 3.5%. mRNA vaccines for COVID-19 have been rapidly designed and manufactured based on the SARS-CoV-2 sequence. However, their deployment outside of a few wealthy countries has been slowed by manufacturing bottlenecks and dependence on cold-chain storage. To fully vaccinate all populations, it is necessary to manufacture highly thermostable vaccines. Accordingly, new methods of stabilizing biomolecules, such as mRNA vaccines, are required.

### SUMMARY OF THE INVENTION

In one aspect, the present disclosure provides compositions comprising:
i) a polymer or a copolymer and a lipid nanoparticle, wherein the w/w ratio of the polymer or the copolymer to the lipid nanoparticle is at least 100:1; or
ii) a polymer or a copolymer and a biomolecule, wherein the w/w ratio of the polymer or the copolymer to the biomolecule is at least 1000:1.

In another aspect, the present disclosure provides microneedle arrays comprising the compositions disclosed herein.

In yet another aspect, the present disclosure provides methods of delivering a therapy to a subject, comprising contacting the subject with the compositions or microneedle arrays disclosed herein.

### BRIEF DESCRIPTION OF THE DRAWINGS

FIG. 1A shows protein microneedle tip-loading using the one-step or two-step fabrication method.

FIG. 1B shows DNA and protein (bovine serum albumin (BSA)) loading in microneedle patches (MNPs) made by hand or using a vaccine printer.

FIG. 1C is a schematic of lipid nanoparticles (LNPs) and their composition after mixing with the MNPs polymer formulation.

FIG. 1D is a graph showing the luminescence of fresh formulations of polymer:LNP preparations. LNPs encapsulating mRNA that encodes for the firefly luciferase (fLuc) were mixed with various water-soluble polymers, and then dried. The formulation was dissolved in PBS, then HeLa cells were transfected. Protein expression was measured 24 h after transfection and results are presented as percent of protein expression obtained with fresh LNPs in PBS.

FIG. 1E shows the chemical structures of two different ionizable lipids used for LNPs containing fLuc mRNA.

FIG. 1F is a schematic description of the needle dissolution and cargo delivery to the intradermal space.

FIG. 1G is a graph showing the luminescence six hours after footpad application via MNP in mice for LNPs composed of two different ionizable lipids. LNPs made with Lipid 5 show a strong response to a dose containing 1 μg of fLuc mRNA.

FIG. 1H shows a comparison of intramuscular and microneedle patch administration for Lipid 5 LNPs containing 1 μg of fLuc-encoding mRNA. MNP administration produces significantly higher luminescence for an equivalent dose.

FIG. 2A shows that pyramid MNPs made from PVP:PVA fail at higher forces than conical MNPs from the same materials.

FIG. 2B shows that pyramid MNPs made from PVP:PVA are stiffer than conical MNPs made from the same materials.

FIG. 2C shows that both conical and pyramid MNPs are capable of penetrating (black arrow) the epidermis (e) and accessing the dermis (d) for intradermal delivery.

FIG. 2D are images of the pyramid and conical MNPs at 0 minutes and 10 minutes after contact with the skin. Pyramid MNPs dissolve significantly faster than conical MNPs.

FIG. 2E is a graph showing the antibody titer of mice vaccinated using either an intramuscular injection (IM) or the microneedle patch.

FIG. 2F shows anti-S protein IgG titers at day 49 (one week post-boost) in mice immunized with MNPs or IM injections containing various SARS-CoV-2 S protein vectors.

FIG. 2G shows binding titers against various S protein variants, before (3 weeks) and after (9 weeks) the boost dose.

FIG. 2H shows that PVP:PVA stabilizes mRNA-LNPs in MNPs. Protein expression remains high after being stored for six months at room temperature. Conversely, protein expression of the IM formulation had greatly decreased by the six-month mark.

FIG. 2I shows the dose of mRNA deliverable using microneedle patches with various geometries, in comparison to clinically available mRNA vaccines.

FIG. 3 shows that LNP size is unaffected by concentration and mixing with polymer to form the ink. However, after drying to form an MNP, and then re-dissolving the solid matrix in water to simulate application in vivo, LNP size increases but is still in a range that is viable for cell uptake and efficient translation of mRNA (generally <250 nm).

FIGS. 4A-4C are transmission electron microscopy (TEM) images that show mRNA-LNPs with size 50-100 nm are present in microneedle patches and without irreversible formation of large LNP aggregates.

FIG. 5 shows that the majority of mRNA is contained within a peak centered on approximately ˜1900 nucleotides, demonstrating that the stabilizing polymer not only preserves mRNA-LNP structure, but does not affect the quality of the mRNA preserved with the mRNA-LNP.

FIG. 6 shows an exemplary microneedle printing process.

FIG. 7 shows the effect of ink printing effects on microneedle patches. The graph shows how the hypothesized boundary layer thicknesses would affect the overall bioactivity of the MNP at different patch sizes.

FIG. 8 shows the effect of patch size on luminescence of the MNPs.

FIG. 9 shows the effect of printer alignment on luminesce of the MNPs.

FIG. 10 shows the effect of the size of the dispensing needle on the luminescence of the MNPs.

FIG. 11 shows the effect of the concentration of the ink on the luminescence of the MNPs.

FIG. 12A shows an exemplary backing step method for the synthesis of MNPs.

FIG. 12B shows the effect of backing on the luminescence of the MNPs.

FIGS. 13A & 13B show images of exemplary MNPs.

FIG. 14 shows that PVP-PVA MNP outperform MNP made from other materials.

FIGS. 15A & 15B show the stability of microneedle patches containing mRNA-LNPs.

FIGS. 16A & 16B show the change in size and polydispersity index (PDI) of LNPs over time.

### DETAILED DESCRIPTION OF THE INVENTION

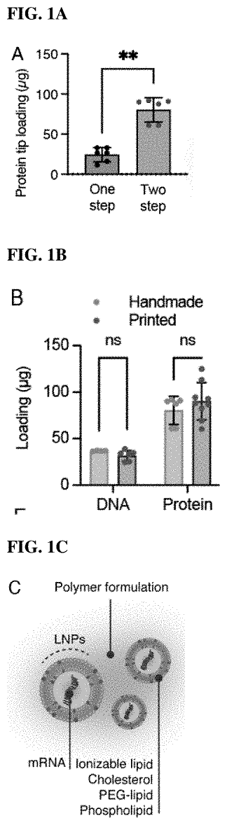

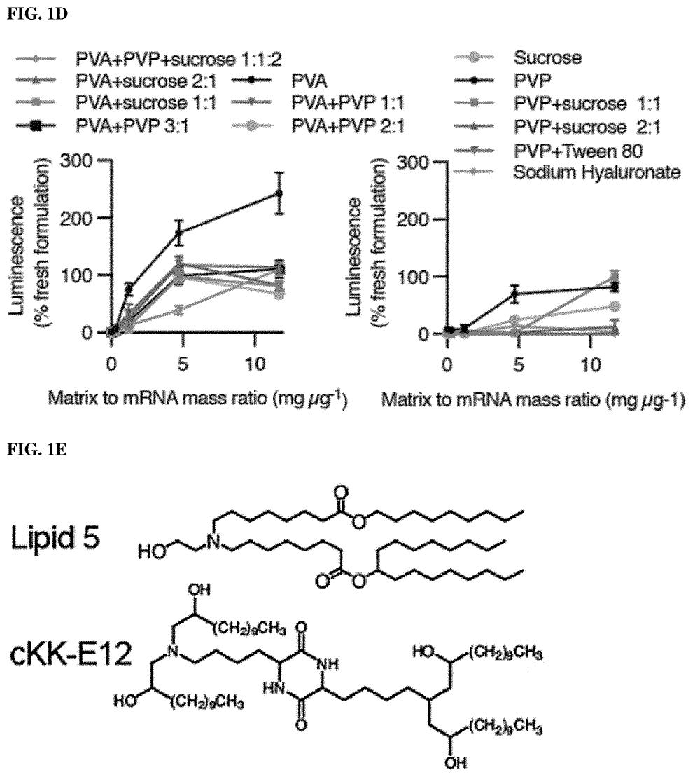

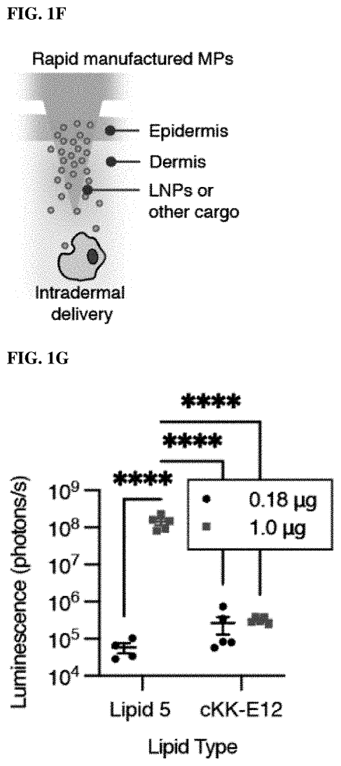

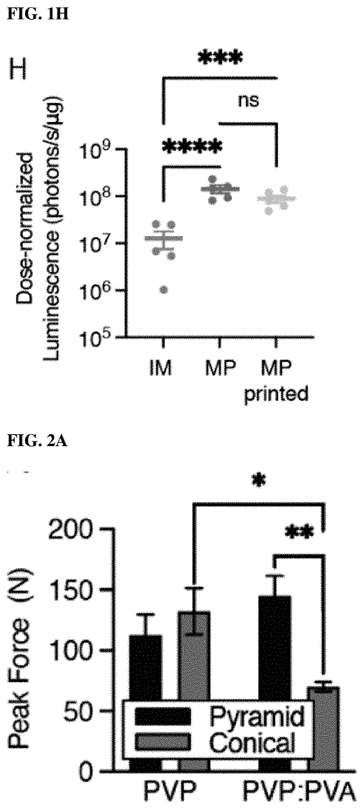

Successful loading of mRNA-loaded LNPs into MNPs has not been reported to date. LNPs are especially difficult to dry in a solid matrix because both chemical and colloidal stability must be preserved. Yet, the volume of polymer available in the needles of MNPs is limited, making it challenging to prevent LNP aggregation. The ideal polymer matrix for MNP fabrication should thus not only preserve LNP integrity after drying, but do so in a minimal volume. To investigate dissolvable polymers for stabilizing LNPs, various biocompatible polymer matrices that are commonly used to fabricate dissolvable microneedles were compared based on their ability to maintain LNP stability at decreasing polymer-to-mRNA mass ratios (i.e., decreasing polymer mass/volume). Negatively charged LNPs of 140 nm diameter and encapsulating 60% of mRNA encoding for the firefly luciferase (fLuc) were fabricated using ionizable lipid, phospholipid, cholesterol and pegylated lipid. LNPs were then mixed with various soluble polymers and dried. After redissolution of the dry matrix, LNPs were used to transfect HeLa cells, and both cell viability and fLuc expression were measured relative to fresh, undried LNPs. None of the formulations tested significantly impaired cell viability. Formulations containing more than 25% PVA relative to PVP were found to be preferable for stabilizing LNPs (FIG. 1C). PVA's slow drying time and elasticity suggest that a preferable formulation is a mixture of PVA with PVP, a fast-drying polymer with good mechanical properties.

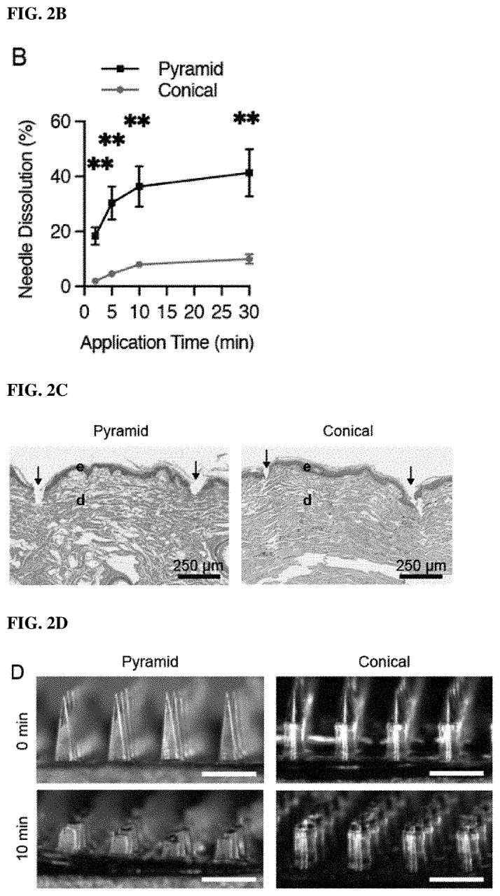

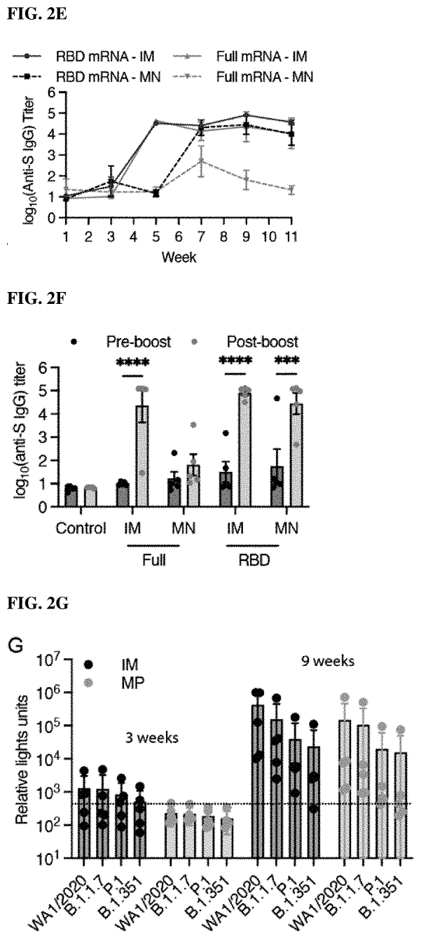

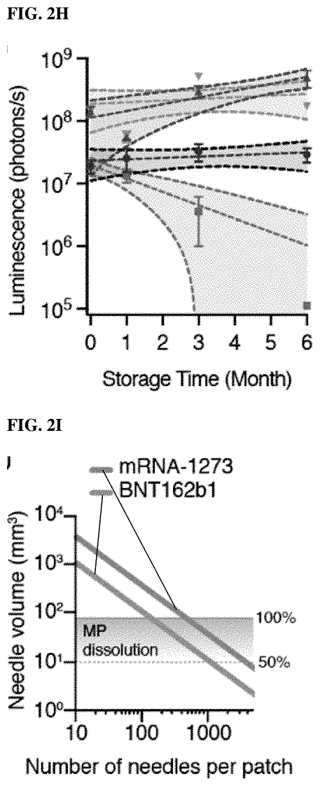

The PVP:PVA blend that was developed for microneedle mRNA-LNP delivery was compared to a conventional mRNA-LNP suspension administered IM at the same dose, using fLuc mRNA as a model for protein expression in mice. Both were stored at room temperature and at 4° C. and assessed after 1, 3, and 6 months. While the IM suspension's potency decreases over 6 months, the MNPs produce consistently high luminescence over the entire storage period (FIG. 2H), even when stored at room temperature. MNPs also maintained high luminescence when stored at 37° C. for one month. This indicates that the formulations could potentially be stored at room temperature and used for immunizations months after fabrication, a drastic improvement in the deliverability of mRNA vaccines, which must currently be held at −60 to −80° C. for long-term storage.

In one aspect, the present disclosure provides compositions comprising:
i) a polymer or a copolymer and a lipid nanoparticle, wherein the w/w ratio of the polymer or the copolymer to the lipid nanoparticle is at least 100:1; or
ii) a polymer or a copolymer and a biomolecule, wherein the w/w ratio of the polymer or the copolymer to the biomolecule is at least 1000:1.

In certain embodiments, the composition comprises a polymer or a copolymer and a lipid nanoparticle, wherein the w/w ratio of the polymer or the copolymer to the lipid nanoparticle is at least 100:1.

In certain embodiments, the w/w ratio of the polymer or the copolymer to the lipid nanoparticle is about 100:1, 150:1, 200:1, 250:1, 300:1, 350:1, 400:1, 450:1, or 500:1. In certain embodiments, the w/w ratio of the polymer or the copolymer to the lipid nanoparticle is about 333:1.

In certain embodiments, the lipid nanoparticle comprises an ionizable lipid, cholesterol, a polyethyleneglycol lipid, a phospholipid, or a combination thereof. In certain embodiments, the lipid nanoparticle comprises 1-octylnonyl 8-[(2-hydroxyethyl)[8-(nonyloxy)-8-oxooctyl]amino]octanoate (i.e., lipid 5, CAS Ref. No.: 2089251-33-0). In certain embodiments, the lipid nanoparticle consists essentially of 1-octylnonyl 8-[(2-hydroxyethyl)[8-(nonyloxy)-8-oxooctyl]amino]octanoate (i.e., lipid 5). In certain embodiments, the lipid nanoparticle comprises 3,6-bis(4-(bis(2-hydroxydodecyl)amino)butyl)piperazine-2,5-dione (i.e., cKK-E12). In certain embodiments, the lipid nanoparticle consists essentially of 3,6-bis(4-(bis(2-hydroxydodecyl)amino)butyl)piperazine-2,5-dione (i.e., cKK-E12).

In certain embodiments, the diameter of the lipid nanoparticle is about 50 nm, about 75 nm, about 100 nm, about 125 nm, about 150 nm, about 175 nm, about 200 nm, about 225 nm, about 250, about 275 nm, or about 300 nm. In certain embodiments, the diameter of the lipid nanoparticle is about 125 nm. In certain embodiments, the diameter of the lipid nanoparticle is about 225 nm.

In certain embodiments, the composition further comprises a biomolecule. In certain embodiments, the w/w ratio of the polymer or the copolymer to the biomolecule is about 600:1, about 700:1, about 800:1, 900:1, or about 1000:1. In certain embodiments, the w/w ratio of the polymer or the copolymer to the biomolecule is at least 1000:1. In certain embodiments, the w/w ratio of the polymer or the copolymer to the biomolecule is about 1,250:1, about 1500:1, about 1750:1, about 2000:1, about 2,250:1, about 2,500:1, about 2,750:1, or about 3000:1. In certain embodiments, the w/w ratio of the polymer or the copolymer to the biomolecule is about 4,000:1, about 5,000:1, about 6,000:1, about 7000:1, about 8,000:1, about 9,000:1, or about 10,000:1.

In certain embodiments, the composition comprises a polymer. In certain embodiments, the polymer is polyvinylpyrrolidone. In certain embodiments, the polymer is polyvinylalcohol.

In certain embodiments, the copolymer comprises a plurality of repeat units of vinylalcohol; a plurality of repeat units of vinylpyrrolidinone; and the copolymer is a block copolymer or a random copolymer. In certain embodiments, the copolymer consists essentially of a plurality of repeat units of vinylalcohol; and a plurality of repeat units of vinylpyrrolidinone; and the copolymer is a block copolymer or a random copolymer.

In certain embodiment, the mass ratio of polyvinylalcohol to polyvinylpyrrolidone is about 1:1, about 2:1, about 3:1, about 4:1, about 5:1, or about 6:1. In certain embodiments, the mass ratio of polyvinylalcohol to polyvinylpyrrolidone is about 1:1, about 2:1, or about 3:1. In certain embodiments, the mass ratio of polyvinylalcohol to polyvinylpyrrolidone is about 1:1.

In certain embodiments, the copolymer comprises a plurality of repeat units of vinylalcohol; a plurality of repeat units of sucrose; and the copolymer is a block copolymer or a random copolymer. In certain embodiments, the copolymer consists essentially of a plurality of repeat units of vinylalcohol; a plurality of repeat units of sucrose; and the copolymer is a block copolymer or a random copolymer. In certain embodiments, the mass ratio of polyvinylalcohol to sucrose is about 1:1, about 2:1, about 3:1, about 4:1, about 5:1, or about 6:1. In certain embodiments, the mass ratio of polyvinylalcohol to sucrose is about 1:1 or about 2:1.

In certain embodiments, the copolymer comprises a plurality of repeat units of vinylalcohol, a plurality of repeat units of polyvinylpyrrolidone, a plurality of repeat units of sucrose; and the copolymer is a block copolymer or a random copolymer. In certain embodiments, the copolymer consists essentially of a plurality of repeat units derived from vinylalcohol, a plurality of repeat units derived from polyvinylpyrrolidone, a plurality of repeat units derived from sucrose; and the copolymer is a block copolymer or a random copolymer. In certain embodiments, the mass ratio of polyvinylalcohol to polyvinylpyrrolidone to sucrose is about 1:1:1, about 1:1:2, or about 1:1:3. In certain embodiments, the mass ratio of polyvinylalcohol to polyvinylpyrrolidone to sucrose is about 1:1:2.

In certain embodiments, the copolymer is a block copolymer. In certain embodiments, the copolymer is a random copolymer.

In certain embodiments, the polymer comprises 50-500 repeat units. In certain embodiments, the polymer comprises 50-250 repeat units. In certain embodiments, the polymer comprises 75-125 repeat units. In certain embodiments, the polymer comprises about 70, about 80, about 90, about 100, about 110, or about 120 repeat units. In certain embodiments, the polymer comprises about 90 repeat units. In certain embodiments, the copolymer comprises 250-1,500 repeat units. In certain embodiments, the copolymer comprises 500-1250 repeat units. In certain embodiments, the copolymer comprises 600-800 repeat units. In certain embodiments, the copolymer comprises about 600, about 650, about 700, about 750, about 800, about 850, or about 900 repeat units. In certain embodiments, the copolymer comprises about 700 repeat units.

In certain embodiments, the biomolecule is a protein. In certain embodiments, the biomolecule is an ribonucleic acid (RNA). In certain embodiments, the RNA is mRNA, circular RNA, ribosomal RNA, small nuclear RNA, microRNA, long non-coding RNA, inhibitory RNA, small interfering RNA, or transfer RNA. In certain embodiments, the biomolecule is a DNA.

In certain embodiments, the concentration of the biomolecule is about 50 μg/mL, about 100 μg/mL, about 150 μg/mL, about 200 μg/mL, about 250 μg/mL, about 300 μg/mL, about 350 μg/mL, about 400 μg/mL, about 450 μg/mL, about 500 μg/mL, about 550 μg/mL, about 600 μg/mL, about 650 μg/mL about 700 μg/mL, about 750 μg/mL, about 800 μg/mL, about 850 μg/mL, about 900 μg/mL, about 950 μg/mL or about 1,000 μg/mL. In certain embodiments, the concentration of the biomolecule is about 650 μg/mL, about 700 μg/mL, or about 750 μg/mL. In certain embodiments, the concentration of the biomolecule is about 700 μg/mL.

In certain embodiments, the biomolecule has improved stability (e.g., improved thermostability) as compared to the biomolecule alone or the biomolecule in a composition not disclosed herein.

In certain embodiments, the composition further comprises a pharmaceutically acceptable excipient.

In another aspect, the present disclosure provides a microneedle array comprising the compositions disclosed herein.

In certain embodiments, the microneedle array is in the form of a patch.

In certain embodiments, the microneedle array dissolves in about 5-15 minutes after contacting an epidermis (e.g., a human epidermis).

In certain embodiments, the microneedles are pyramidal. In certain embodiments, the microneedles are pyramidal.

In certain embodiments, the microneedle array comprises about 40, about 50, about 60, about 70, about 80, about 90, or about 100 needles. In certain embodiments, the microneedle array comprises about 40, about 50, or about 60 needles. In certain embodiments, the microneedle array comprises about 40 or about 50 needles.

In certain embodiments, the microneedle array is formed without backing.

In certain embodiments, the microneedle array is formed using a single drying cycle.

In yet another aspect, the present disclosure provides a single-injection device comprising a composition disclosed herein. In yet another aspect, the present disclosure provides a pulsatile-release microdevice comprising a composition disclosed herein. Exemplary single-injection and pulsatile-release microdevices are disclosed in U.S. Pat. Nos. 10,300,136, 10,960,073, US 2021/0205444 A1, and US 2019/0076631 A1, the contents of each of which are incorporated by reference herein.

In yet another aspect, the present disclosure provides a polymeric device comprising a polymeric shell and at least one discrete region comprising a composition disclosed herein, optionally in combination with a stabilizing excipient, wherein the shell and discrete regions are formed from successive layers of polymeric particles bonded together by solvent and/or temperature by three dimensional printing or micromolding.

In certain embodiments, the device is formed by three dimensional printing or micromolding of a biocompatible polymer, the device comprising a polymeric shell and one or more discrete regions comprising a composition disclosed herein, optionally in combination with a stabilizing excipient for a composition disclosed herein, wherein an effective amount of a composition disclosed herein is released in two or more time periods to elicit an immune response, and with insufficient release of the composition disclosed herein between the release periods to elicit an immune response in vivo.

In certain embodiments, the device is made by three dimensional printing.

In certain embodiments, the device further comprises an effective amount of a composition disclosed herein to elicit an immune response in vivo which is present in a stabilizing excipient, on the surface of the device, or mixed with the polymer of the device.

In certain embodiments, the device comprises a stabilizing excipient selected from the group consisting of sugars, oils, lipids, and carbohydrates.

In certain embodiments, a composition disclosed herein elicits an immune response to an infectious agent or to a tumor. In certain embodiments, the composition elicits an immune response to an infectious agent; and the infectious agent is a virus, bacteria, fungus or protozoan. In certain embodiments, the infectious agent is a virus; and the virus is selected from the group consisting of polio, influenza, hepatitis, rotavirus, measles, mumps, rubella, and varicella. In certain embodiments, the composition elicits an immune response to a tumor. In certain embodiments, a composition disclosed herein elicits a T cell response to a tumor.

In certain embodiments, the polymer is biodegradable by hydrolysis.

In certain embodiments, the device provides release at intervals of from ten to ninety days.

In certain embodiments, the composition disclosed herein is encapsulated in polymeric particles in the form of microparticles, microcapsules, or microspheres.

In certain embodiments, the device is injectable. In certain embodiments, the device is implantable. In certain embodiments, the device can be applied to a mucosal surface selected from the group consisting of nasal, pulmonary, oral, vaginal and rectal mucosal surfaces.

In certain embodiments, the stabilizing excipient comprises sugar, wherein the sugar is selected from the group consisting of sucrose, trehalose, and combinations thereof. In certain embodiments, the stabilizing excipient comprises monosodium glutamate (MSG). In certain embodiments, the stabilizing excipient comprises magnesium chloride (MgCl₂). In certain embodiments, the sugar comprises sucrose.

In certain embodiments, the device provides release at intervals of from 30 to 60 days.

In certain embodiments, the composition disclosed herein is released in at least three time periods at intervals of from ten to ninety days.

In certain embodiments, the composition disclosed herein is released in at least four time periods at intervals of from ten to ninety days.

In certain embodiments, the device further comprises a buffering agent. In certain embodiments, the buffering agent is selected from the group consisting of magnesium hydroxide, aluminum hydroxide, and myristic acid.

In yet another aspect, the present disclosure provides a microdevice having dimensions of less than one centimeter comprising a micromolded fillable polymeric shell comprising a non-photoactivatable biocompatible polymer having a complex channel or core therein; wherein the channel or core contains a composition disclosed herein.

In certain embodiments, the microdevice further comprises a cap sealing the channel or core.

In certain embodiments, the polymer comprises a biocompatible polymer selected from the group consisting of biodegradable polymers, phase-change polymers, thermoplastic polymers, and combinations thereof.

In certain embodiments, the microdevice dimensions are from about 1 micrometer (μm) to about 1000 μm; and wherein the channel or core dimensions are less than 800 μm.

In certain embodiments, the microdevice has a loading capacity from 1 percent weight/weight (% w/w) to 50% w/w.

In certain embodiments, the volume of the hollow core allows for loading a composition disclosed herein at from 1 percent (%) to 50% of the total volume of the microdevice.

In certain embodiments, the composition disclosed herein is released from discrete regions of the microdevice with different release kinetics.

In certain embodiments, the disclosed herein is released in a defined time period in an amount effective to enhance or tolerize an immune response in vivo.

In certain embodiments, the microdevice further comprises an excipient selected from the group consisting of sugars, salts, oils, lipids, and carbohydrates.

In yet another aspect, the present disclosure provides methods of delivering a therapy to a subject, comprising contacting the subject with the compositions, microneedle arrays, single injection device, pulsatile-release microdevice, device, or microdevice disclosed herein. In certain embodiments, the therapy is a vaccine. In certain embodiments, the therapy is an mRNA vaccine.

### Definitions

Unless otherwise defined herein, scientific and technical terms used in this application shall have the meanings that are understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a composition or an agent to a subject can be carried out using one of a variety of methods known to those skilled in the art. For example, a composition or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A composition or agent can also appropriately be introduced by rechargeable or biodegradable polymeric devices or other devices, e.g., patches and pumps, or formulations, which provide for the extended, slow or controlled release of the composition or agent. Administering can also be performed, for example, once, a plurality of times, and/or over one or more extended periods.

Appropriate methods of administering a substance, a composition or an agent to a subject will also depend, for example, on the age and/or the physical condition of the subject and the chemical and biological properties of the composition or agent (e.g., solubility, digestibility, bioavailability, stability and toxicity). In some embodiments, a composition or an agent is administered orally, e.g., to a subject by ingestion. In some embodiments, the orally administered composition or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compositions can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any nontoxic organic or inorganic salt of any base. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used.

The term “pharmaceutically acceptable basic addition salt” as used herein means any nontoxic organic or inorganic base addition salt of an acid. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition is preferably administered as a pharmaceutical composition comprising, for example, a composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a composition such as a composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a composition of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example, as a patch applied to the skin); and topically (for example, as a cream, ointment or spray applied to the skin). The composition may also be formulated for inhalation. In certain embodiments, a composition may be simply dissolved or suspended in sterile water. Details of appropriate routes of administration and compositions suitable for same can be found in, for example, U.S. Pat. Nos. 10,300,136, 10,960,073, 6,110,973, 5,763,493, 5,731,000, 5,541,231, 5,427,798, 5,358,970 and 4,172,896; published U.S. Patent Application Nos: 2021/0205444 A1, and 2019/0076631 A1; and unpublished U.S. Patent Application No. 63/284,519 and Ser. No. 17/689,314; as well as in patents cited in any of the foregoing, the contents of all of which are incorporated by reference herein in their entirety.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a composition of the invention, with the carrier and, optionally, one or more accessory ingredients. In general, the formulations are prepared by uniformly and intimately bringing into association a composition of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a composition of the present invention as an active ingredient. Compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches, microdevices, and inhalants. In certain embodiments, the dosage form may be in the form of a microparticle or a nanoparticle. The active composition may be mixed under sterile conditions with a pharmaceutically acceptable carrier, and with any preservatives, buffers, or propellants that may be required.

The ointments, pastes, creams and gels may contain, in addition to an active composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a composition of the present invention to the body. Such dosage forms can be made by dissolving or dispersing the composition in the proper medium. Absorption enhancers can also be used to increase the flux of the composition across the skin. The rate of such flux can be controlled by either providing a rate controlling membrane or dispersing the composition in a polymer matrix or gel.

Examples of suitable aqueous and non-aqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a composition at a particular target site.

Actual dosage levels of the active ingredients in the pharmaceutical compositions may be varied to obtain an amount of the active ingredient that is effective to achieve the desired therapeutic response for a particular patient, composition, and mode of administration, without being toxic to the patient.

The selected dosage level will depend upon a variety of factors including the activity of the particular composition or combination of compositions employed, or the ester, salt or amide thereof, the route of administration, the time of administration, the rate of excretion of the particular composition (s) being employed, the duration of the treatment, other drugs, compositions and/or materials used in combination with the particular composition (s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition at levels lower than that required in order to achieve the desired therapeutic effect and gradually increase the dosage until the desired effect is achieved. By “therapeutically effective amount” is meant the concentration of a composition that is sufficient to elicit the desired therapeutic effect. It is generally understood that the effective amount of the composition will vary according to the weight, sex, age, and medical history of the subject. Other factors which influence the effective amount may include, but are not limited to, the severity of the patient's condition, the disorder being treated, the stability of the composition, and, if desired, another type of therapeutic agent being administered with the composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active composition used in the compositions and methods of the invention will be that amount of the composition that is the lowest dose effective to produce a therapeutic effect. Such an effective dose will generally depend upon the factors described above.

If desired, the effective daily dose of the active composition may be administered as one, two, three, four, five, six or more sub-doses administered separately at appropriate intervals throughout the day, composition, in unit dosage forms. In certain embodiments of the present invention, the active composition may be administered two or three times daily. In certain embodiments, the active composition will be administered once daily. In certain embodiments, the active composition will be administered once weekly. In certain embodiments, the active composition will be administered once monthly. In certain embodiments, the active composition will be administered once quarterly.

The patient receiving this treatment is any animal in need, including primates, in particular humans; and other mammals such as equines, cattle, swine, sheep, cats, and dogs; poultry; and pets in general.

In certain embodiments, compositions of the invention may be used alone or conjointly administered with another type of therapeutic agent.

The present disclosure includes the use of pharmaceutically acceptable salts in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation of Exemplary Lipid Nanoparticles

Lipids were dissolved in ethanol at a molar ratio of 35:16:46.5:2.5 (cKK-E12 (Organix):DOPE (Avanti):Cholesterol (Sigma):C14-PEG2000 (Avanti)) or 50:10:38.5:1.5 (Lipid 5(Organix);DOPE:Cholestorol:C14-PEG2000) when using respectively cKK-E12 or Lipid 5 as the ionizable lipid. To prepare the LNPs, the ethanoic solution was rapidly added to and pipette-mixed with a mRNA solution buffered with citrate at pH 3 at volume ratio 3:1 (aqueous:ethanol). The ionizable lipid to mRNA weight ratio was set to 10 and the final mRNA concentration was 0.1 mg mL-1. All nucleic acids were stored at −80° C. and were allowed to thaw on ice prior to use. The LNPs were then dialyzed for at least 2 hours in PBS at 4° C. in a 20,000 MWCO cassette.7 For LNPs in deionized (DI) water, the solution was dialyzed against DI water for an additional minimum of 2 hours at 4° C. When needed, the LNPs were concentrated on an Amicon filter by centrifugation at 3000×g.19 All solutions were kept at 4° C. and used within a week.

mRNA concentration and encapsulation efficiency in the LNPs was estimated using a Quant-iT RiboGreen assay (ThermoFisher) and a modified procedure described elsewhere.7 Briefly, LNPs were diluted in either Tris-EDTA (TE) or TE mixed with triton X-100 buffer (TX). Then, the procedure recommended by the manufacturer was used to quantify the mRNA that is not encapsulated (when diluted with TE) and the total mRNA concentration (when diluted with TX). For size and surface potential, LNPs were diluted 200 times in PBS and measurement was achieved using [REF Malvern]. When measuring the mRNA loading in MNPs, needles were cut, and dissolved in TE and TX. The total mRNA concentration found was used to estimate the encapsulated mRNA concentration based on its encapsulation efficiency.

### Example 2: Preparation of Exemplary Polymer:Lipid Nanoparticle Formulations

Polymers were solubilized in PBS or DI water at a concentration ranging from 10% to 30% w/w depending on their solubility. These solutions were then weighted and mixed with LNPs solution to reach the appropriate polymer to mRNA mass ratio. The mixture was immediately dried in a Low-Bind Eppendorf tube in a desiccator under −0.5 bar vacuum. After 24 hours drying, the pellet was redissolved in PBS and incubated for 10 min at 37° C. The PBS volume used was adjusted so that 15 μL of solution contains 50 ng of encapsulated mRNA. This solution was used to transfect cells.

### Example 3: Quantification of Properties of Exemplary Polymer:Lipid Nanoparticle Formulations

### Transfection Study

HeLa cells were cultured in high glucose Dulbecco's Modified Eagles Medium with phenol red (DMEM, Invitrogen) supplemented with 10% FBS (Invitrogen) and 1% antibiotic (Invitrogen). 10,000 cells were seeded in wells of a white 96-well plate in full growth medium. 20 hours after seeding, 15 μL of fresh LNPs or dissolved formulation were added to the growth medium. In all cases, 50 ng of encapsulated mRNA were added to each well. 24 hours after transfection, 100 μL of Bright-Glo Luciferase Assay Kit (Promega) reagent was added and luminescence was measured in the following 2 minutes using a Tecan multiplate reader.

### Example 4: Fabrication of Exemplary Microneedle Patches (MNPs)

MNPs were fabricated by loading and drying 200 μL of a 20% w/w PVP, PVA or PVP:PVA solution (in PBS or DI water) in a PDMS mold. Different drying methods were tested in this paper. When LNPs, DNA or protein were loaded in the MNP, a two-step loading procedure was used. First, 200 μL of a solution containing 0.8, 1.6, 2 or 8 mg of PVP:PVA and various amounts of LNPs (expressed as encapsulated mRNA mass), protein or DNA was loaded and dried. Then, a PVP:PVA (20% w/w in PBS or DI water) was used to bring the total MNP mas to 40 mg of PVP:PVA. That polymer solution (volume ranging between 160 and 196 μL) was loaded and dried.

### Example 5: Properties of Exemplary Microneedle Patches

### Measurement of Loading Time

The time needed to load the liquid formulation in the PDMS negative mold was evaluated by recording the loading using a camera. The camera was placed on the side of the PMDS mold, and the focus was set on the microneedles through the transparent PDMS. For this experiment, single PDMS molds were used rather than the 5×5 PDMS sheet used elsewhere. These single molds were fabricated in Petri dishes, which allows to have clear sides.

Co-Loading Multiple Vaccines with Dye

Vaccine that dries in the backing of the microneedle patch creates additional vaccine waste. To concentrate vaccine in the microneedle tips, a two-step tip-loading process was developed using an automated robotic equipment for printing MNPs, called mobile vaccine printer (MVP). First, the vaccine is loaded into the mold and dried with a minimal amount of polymer. Then, polymer only is loaded and dried to form the backing. The MVP can also be used to tip-load two cargos simultaneously demonstrated by red and blue dye. The resulting patches showed excellent feature size and tip-loading, evident by higher concentration of the dyes at the tips.

### Simulation of Flow Inside PDMS Molds

Flow of polymer solution into an individual microneedle cavity was simulated by CFD module in COMSOL Multiphysics® V 5.2. The boundary condition was specified based on a Poiseuille (pressure-driven) flow and an effective pressure difference was allocated between the inlet (microneedle head), and outlet (microneedle tip). The effective pressure gradient was used to empirically capture the resulting pressure interaction between external pressure applied by pump (contributing to flow), and the capillary pressure (resisting the flow). The pressure gradient was calibrated based on experimental observation of filling of PDMS by polymer solution. As such, the value of effective pressure gradient was selected such that for a given viscosity of the polymer solution, the resulting average velocity magnitude was comparable to experimental velocity of polymer solution. One the effective pressure gradient was approximated, the effect of different design parameters was simulated using CFD module in COMOSL. The model was parametrized to study the effect of various design parameters on the average velocity magnitude of the polymer solution in a simulated needle. These parameters include viscosity and density of the polymer solution, ratio of conical/cylindrical segment in microneedle, needle diameter, total needle length, and pressure gradient at the inlet/outlet. Velocity magnitude was the key output as it determines the approximate filling time after applying vacuum. Simulations were performed in steady state conditions, by including the effect of gravity, and physically optimized mesh structure.

### Dispensing Optimization

A design of experiment (DOE) approach was followed to study the effect of various design parameters on the collective size of the drops dispensed from the robotic nozzle (needle), as the output response. To this end, an L₁₈ orthogonal array design of experiment was constructed in Minitab. The effect of following design parameters was studied: 1) attachment of a 0.2 μm filter to the nozzle, 2) needle gauge, 3) dispensing height, 4) dispensing flow rate, 5) polymer viscosity by changing polymer composition. Results (n=3-5) were analyzed in Minitab to find the mean response of drop area as a function of change these parameters. The total dispensed volume was considered constant equal to 200 μL. Different flowrates were achieved by changing the dispensing time but retaining total volume constant.

### Protein Quantification

Needles of a MNP loaded with BSA were cut and dissolved in DI water. A BCA (ThermoFisher) was used to quantify the protein loading using the standard procedure for a 96-well plate. An internal standard of PVP and PVA was added to the calibration curve to account for any PVP or PVA interference with the assay.

### Water Content Analysis

The water content of the fabricated MNPs at different time points and stages of drying was quantified by Thermogravimetric Analysis (TGA) using a Pyris 1 Thermogravimetric analyzer (PerkinElmer) with heating rate at 20° C./min from 50 to 600° C. under nitrogen flow (20 mL/min). The water content was evaluated by analyzing only the needles of the MNPs placed in ceramic pans and not the backing. All analyses were conducted in triplicate.

### Compressive Mechanical Testing

For compression testing, a single MNP was mounted between compression platens (Instron 2501 Series) and compressed at a rate of 1 mm/min using an Instron 5943 with a 500 N load cell. The peak force prior to microneedle failure was reported and the slope of the linear region of initial compression. Microneedle failure mode was determined by imaging the patches after testing.

The mechanical and functional properties of MNPs produced with the combination of polymers preferable for stabilizing LNPs, were evaluated considering both conical and pyramidal needles as potentially viable geometries. In terms of both peak force (FIG. 2A) and stiffness (FIG. 2B), pyramidal MNPs made of PVP:PVA outperform conical MNPs of the same composition. Though all MNPs meet the minimum requirements for piercing skin, PVP:PVA MNPs are generally stronger than MNPs produced using PVP alone. Skin puncture and microneedle dissolution for both PVP:PVA geometries was confirmed with ex vivo pig skin evaluated by image analysis (FIGS. 2C & 2D). While both geometries can access the dermis, pyramidal microneedles dissolve to a significantly greater extent (FIGS. 2E & 2F). Pyramid MNPs were used for further experiments.

### Evaluation of Needle Insertion (Histology)

MNPs were applied ex vivo on pig skin using a mini spring-loaded applicator of high impact (Micropoint Technologies) for 2, 5, 10 or 30 min. Subsequently, the skin samples were fixed into formalin for 48 h and then transferred to ethanol 70% and embedded in paraffin wax. Samples were sectioned and stained with hematoxylin and eosin.

### Needle Dissolution/Volume Delivered

For the quantification of microneedle volume delivery as a function of application time microneedle patches were imaged before and after application ex vivo on pig skin using Leica DFC450. The patches were placed in a transverse manner for imaging using with LAS V4.7 software. Needle length of at least 10 needles for each patch was calculated using Image J and these measurements were performed in triplicate (3 patches applied for each timepoint).

Luciferase mRNA Expression in Mice

All experimental procedures were ethically approved and performed under the guidelines of the Division of Comparative Medicine by Massachusetts Institute of Technology. Six-week to twenty-week-old female C57BL/6 mice (Charles River) were used and monitored for safety. MNPs were fabricated with LNPs carrying pseudouridine-modified mRNA encoding for firefly luciferase (TriLink Biotechnologies) using the two-step loading method described above. LNP dose and ionizable lipid chemistry were varied, maintaining at least a 1000:1 polymer:mRNA mass ratio. Two ionizable lipids that were previously selected for intravenous or intramuscular mRNA administration methods were studied, respectively: cKK-E127 and Lipid 510 (Organix). MNPs were applied to either footpad. To dissolve more fully the full dose of LNPs carried in a MNP, when a 10×10 array was applied, MNPs were divided into two halves and applied consecutively to the same footpad, for 10 minutes per half. As a positive control, LNPs were administered to the caudal thigh muscle as a 40 μL suspension in PBS at various doses.

Six hours after MNP application, mice were imaged for bioluminescence in an IVIS kinetic imaging system (PerkinElmer). 15 minutes prior to imaging, mice were injected intraperitoneally with Rediject D-Luciferin Ultra (PerkinElmer) at 150 mg/kg. Luminescence was quantified using LivingImage software (PerkinElmer).

### DNA Quantification

Needles of a MNP loaded with DNA were cut and dissolved in TE buffer. DNA was quantified using UV-absorption at 260 nm and 280 nm using a Nanodrop. Since PVP also absorbs in UV range, the same amount of PVP was added to the calibration curve to account for its contribution.

### Example 6: Vaccination of Mice Using Exemplary Formulations of the Disclosure

Six-week-old female C57BL/6 mice (Charles River) were used and monitored for safety. MNPs were fabricated with LNPs carrying pseudouridine-modified mRNA encoding for the wild type SARS-CoV-2 spike protein with furin cleavage site deletion, two proline mutations, and a trimerization foldon for stability (TriLink Biotechnologies) using the two-step loading method described above. 5×5 MNP arrays with were fabricated containing a 2 μg dose of mRNA, which was verified by RNA quantification assay. 4 5×5 arrays were applied to the left and right footpad of mice, with a 10 minute application time for each, for a total mRNA dose of 8 μg per mouse. As a positive control, LNPs were administered to the right caudal thigh muscle as a 40 μL suspension in PBS at 10.5 μg per mouse. A p CMV3 DNA plasmid encoding for the wild-type SARS-CoV-2 spike protein was used for DNA MNP vaccinations. DNA was loaded at a 100 μg dose per MNP using the two-step loading method and applied as above. As a positive control, 100 μg dose of the same SARS-CoV-2 DNA plasmid was injected in 40 uL of PBS to the right caudal thigh muscle.

### Example 7: Stability of Exemplary Formulations

MNPs were fabricated with LNPs carrying pseudouridine-modified mRNA encoding for fireflyluciferase (TriLink Biotechnologies) using the two-step loading method described above. mRNA dose was maintained at 1.0 μg per patch, maintaining a constant 1000:1 polymer:mRNA mass ratio for all stability experiments. Lipid 5 (Organix) was used as the ionizable lipid. MNP size and application are identical to above studies of firefly luciferase expression. MNPs were stored in a container with silica desiccant at various temperatures. As a positive control, sealed vials of suspension containing the same amount of firefly luciferase mRNA in LNPs made with Lipid 5 were stored at various temperatures alongside MNPs.

### Example 8: Further Characterization of LNPs and MNPs

It was hypothesized that PVP-PVA stabilizes mRNA-LNPs by allowing them to re-disperse, maintain structure, and retain mRNA bioactivity when the PVP-PVA matrix dissolves. Therefore DLS and TEM was used to assess LNP structure, and capillary electrophoresis was used to assess mRNA integrity. In general, samples were prepared by mixing mRNA-LNPs with PVP-PVA solution to form an “ink”, and then this ink was dried and re-dissolved in water for analysis. In some cases, the structure was captured at each of the different stages of the ink preparation process. The polymer:mRNA mass ratio studied for all experiments in this report is 333:1, unless noted otherwise. Based on in vitro data showing increasing stability at increasing mass ratios, it is assumed that these findings are generalizable to mass ratios greater than 333:1.

Lipid Nanoparticle Size after Stabilization: Dynamic Light Scattering (DLS)

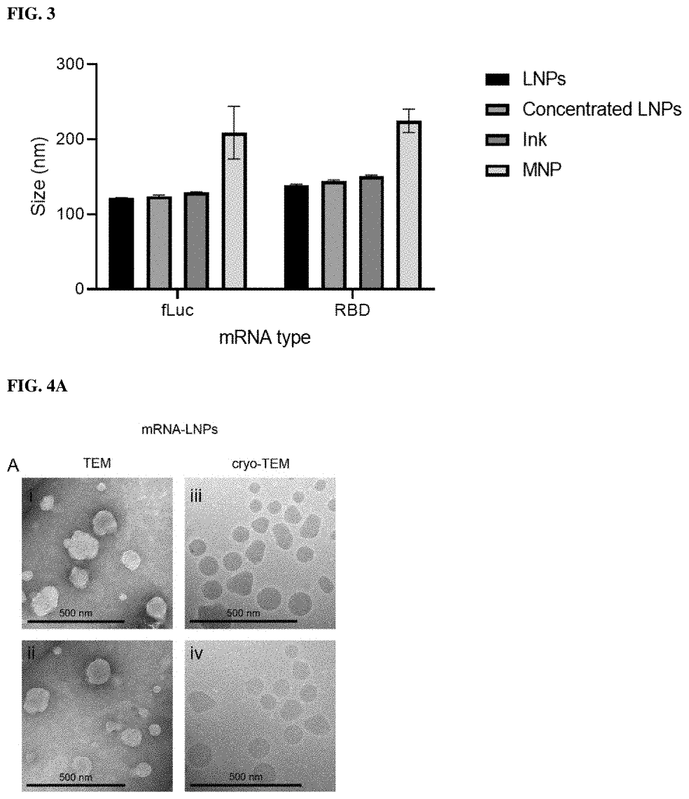

LNP size is unaffected by concentration and mixing with polymer to form the ink (before drying) (FIG. 3). After drying to form an MNP, and then re-dissolving the solid matrix in water to simulate application in vivo, LNP size increases but is still in a range that is viable for cell uptake and efficient translation of mRNA (generally <250 nm). This suggests that the majority of mRNA-LNPs are able re-disperse after MNP dissolution without irreversible formation of large LNP aggregates

Lipid Nanoparticle Structure after Stabilization: Transmission Electron Microscopy (TEM)

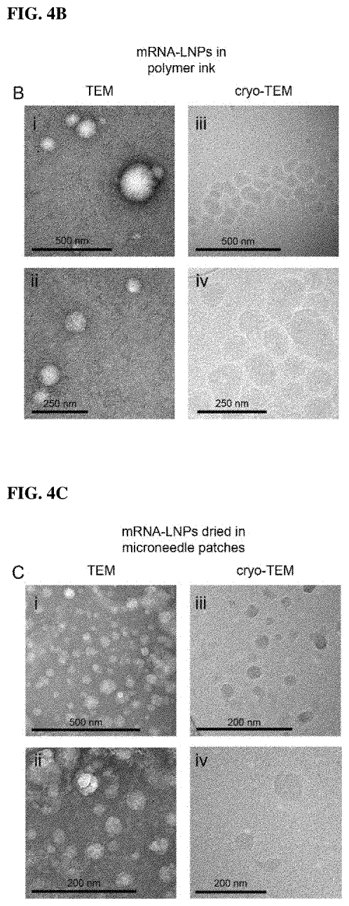

TEM corroborates the above DLS (FIGS. 4A-4C). mRNA-LNPs with size 50-100 nm are present in microneedle patches and without irreversible formation of large LNP aggregates.

mRNA after Stabilization: Fragment Analyzer

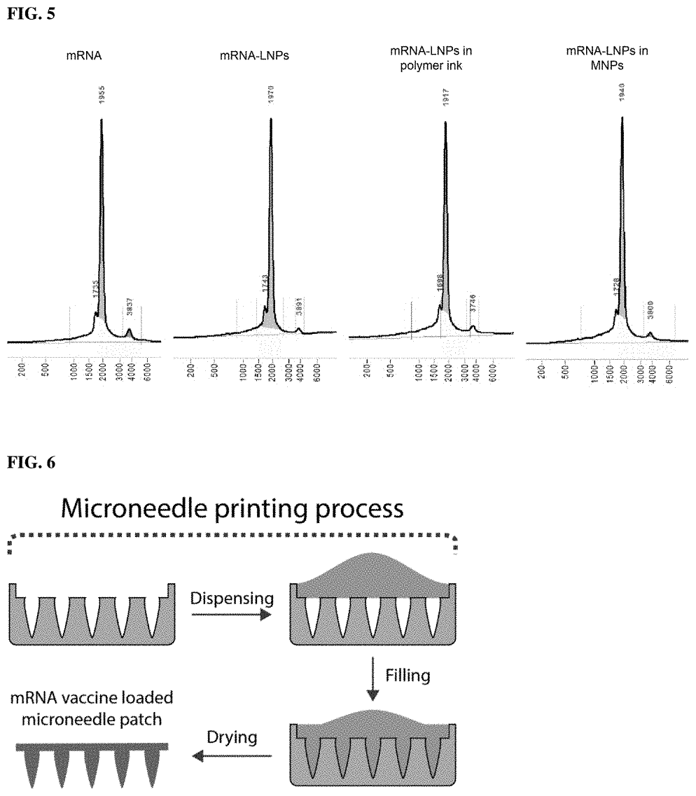

AATI Fragment Analyzer 2 was used to perform capillary electrophoresis on various firefly luciferase mRNA samples in ink or microneedle patches, aiming to determine mRNA size in nucleotides (FIG. 5). Tween solution was used to dissociate mRNA from LNPs before processing using the AATI Fragment Analyzer 2. In all cases, the majority of mRNA is contained within a peak centered on approximately ˜1900 nucleotides, demonstrating that the stabilizing polymer not only preserves mRNA-LNP structure, but does not affect the quality of the mRNA preserved with the mRNA-LNP.

### Example 9: Further Characterization of LNPs and MNPs

Vaccine “ink” comprises of mRNA-LNPs, dissolved stabilizing polymer, and water. It was discovered that the dispensing, drying, and molding of the ink as it forms a subsequent microneedle patch (MNP) can have a significant effect on the bioactivity of the applied microneedle patch in vivo. It has been determined that the ink concentration and cycling of repeated drying can affect in vivo bioactivity.

For all in vivo experiments shown in this section, a single microneedle patch containing mRNA-LNPs encoding for firefly luciferase was applied to the footpad of a C57BL/6 mouse for 10 minutes. Mice were injected i.p. with luciferin substrate and imaged with a PerkinElmer IVIS Spectrum under for luminescence detection, and total luminescence was quantified in the region of interest using PerkinElmer LivingImage software (FIG. 6).

### Ink Printing Effects on Microneedle Patches: Patch Size and Dispensing

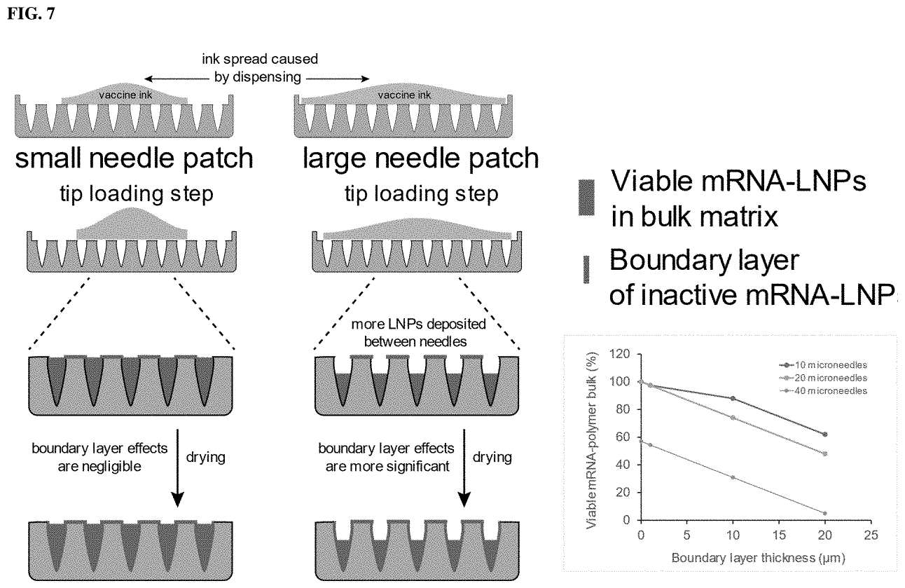

Due to the viscosity of the ink and accuracy of dispensing, microneedle patches can have varying sizes. This causes mRNA-LNPs and stabilizing polymer to spread over a higher surface area, and it was hypothesize that the boundary layer between microneedle patch and mold has low bioactivity due to increased aggregation of mRNA-LNPs. FIG. 7 shows how the hypothesized boundary layer thicknesses would affect the overall bioactivity of the MNP at different patch sizes. MNPs with different sizes were fabricated using ink containing the same amount of mRNA-LNPs to confirm this hypothesis. Patch size alone (as determined by ink spread) can negatively affect bioactivity.

### Ink Printing Effects on Microneedle Patches: Alignment of First and Second Dispensing Step

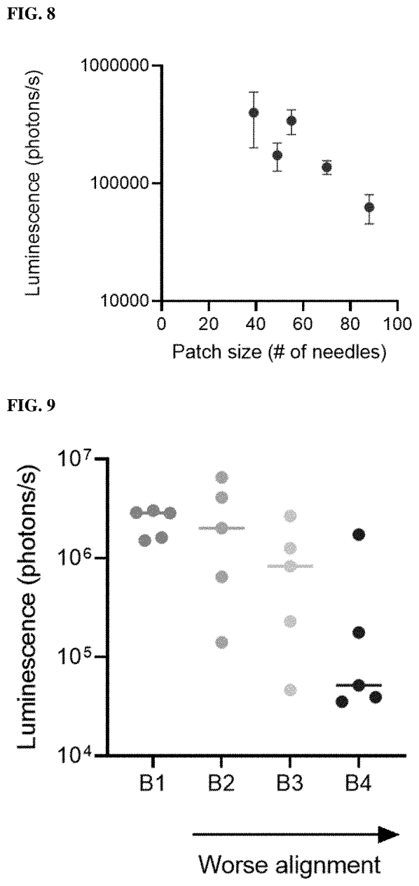

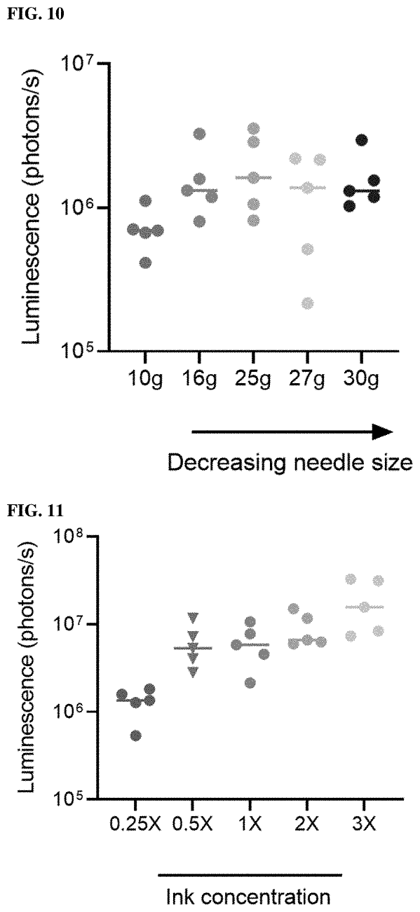

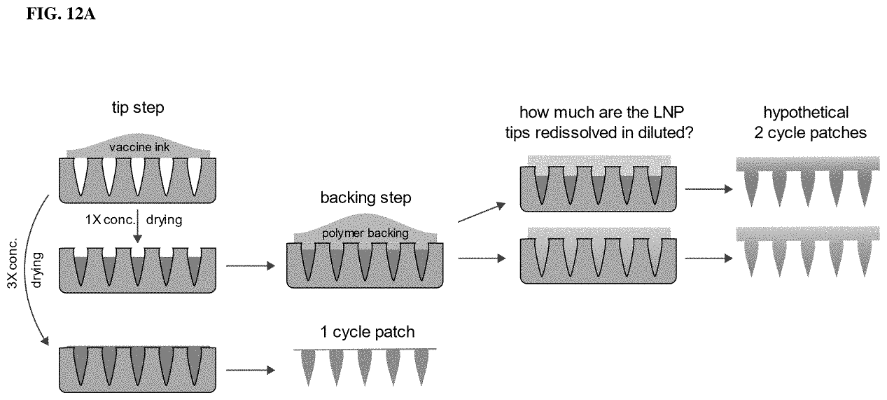

Conventionally, a concentrated water-soluble polymer is dispensed onto tip loaded microneedles to create a patch with a uniform and durable backing. The accuracy of this second dispensing step is critical to making a patch with high bioactivity. LNP were fabricated with increasingly worse alignment but the same size to test this hypothesis (FIG. 8).

### Ink Printing Effects on Microneedle Patches: Dispensing Needle Size

A needle is used to dispense vaccine ink onto the microneedle mold. The needle dispensing the vaccine ink has no significant effects on the stability of mRNA-LNPs (FIG. 9).

### Ink Printing Effects on Microneedle Patches: Concentration of Ink

The concentration of the ink used for printing can be varied. Here polymer and mRNA ratio is held constant, but the concentration of both polymer and mRNA can be increased or decreased proportionally in water to maintain a constant ratio. By increasing or decreasing the concentration of components in the ink, the properties of the ink—such as, but not limited to, viscosity and surface tension—can be manipulated. Likewise, using a lower or higher concentration of solids in the ink, within a constant droplet volume dispensed onto the microneedle mold, can have dramatic effects on the resultant microneedle patch—such as fuller needles or more waste deposited on the intermediate spaces of the mold.

Herein ink concentration from 0.25× (14 mg/mL PVP-PVA, 56 μg/mL mRNA) to 3× (167 mg/mL PVP-PVA, 694 μg/mL mRNA) were varied and the resultant MNP were studied in vivo. All patches received the same layer of polymer backing: 8 mg of PVP-PVA dissolved in 40 μL of water. There was a increasing trend of luminescence signal in vivo with concentration, showing that concentration of the ink can be used to increase protein expression in MNPs (FIG. 11).

### Ink Printing Effects on Microneedle Patches: Backing Deposition and Potential Effects of Multiple Re-Dissolution Cycles

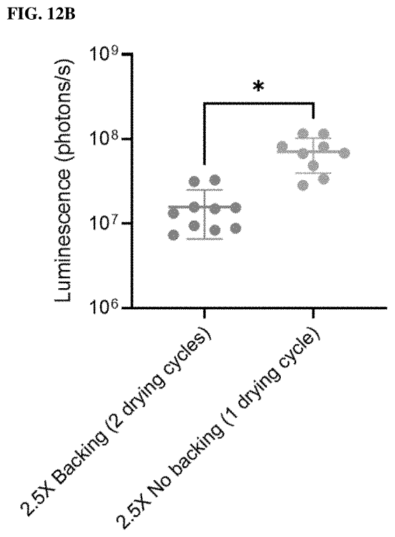

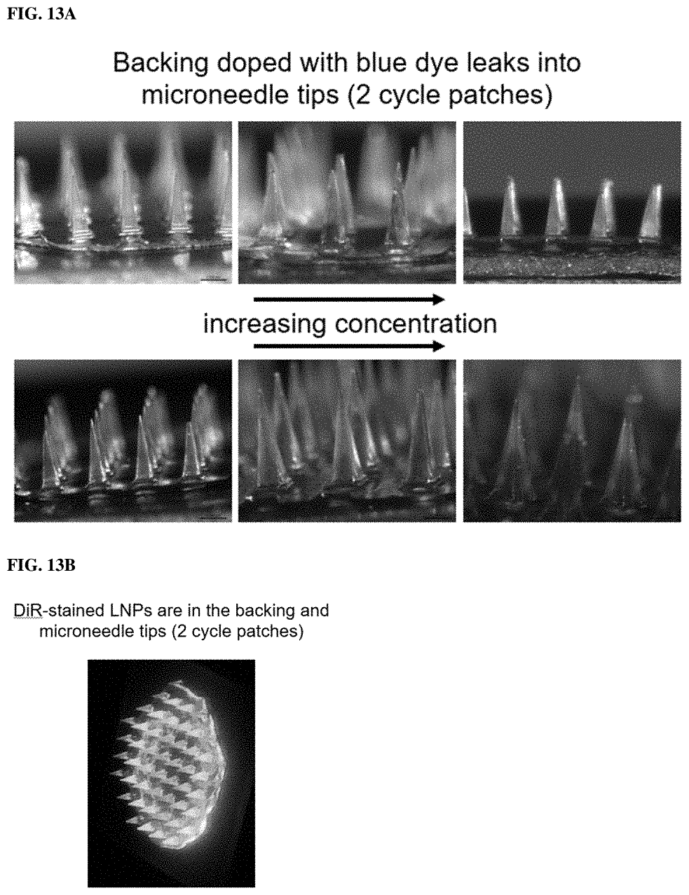

In testing various ink concentrations, it was discovered that mechanically stable MNPs could be fabricated without depositing a backing layer at 3× concentration. In other words, an MNP could be formed using only the tip deposition step at 3×. Applying these in vivo revealed that mRNA-LNPs made using a single tip dispensing cycle are more active than those made using a 2-step tip and back process. Additional tests have shown that 2 cycle patches have significant mixing of the tips and backing (representative dye and dyed-LNP images shown) (FIG. 12A-13B).

Excipient Screen with DLS

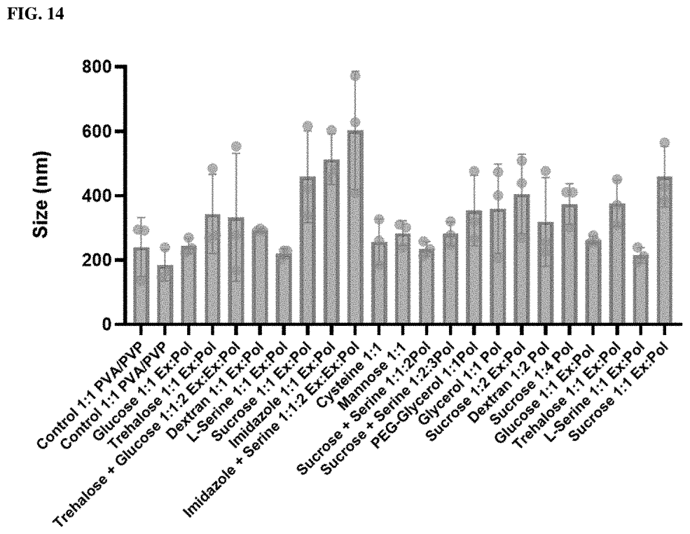

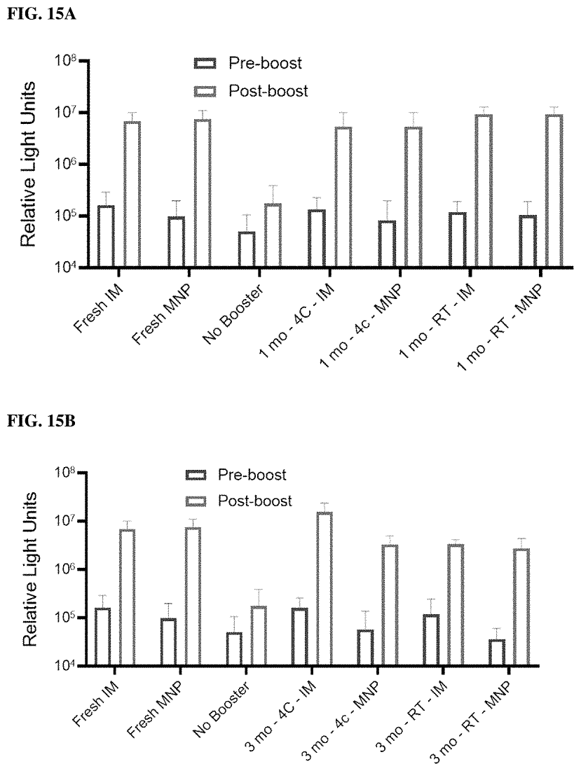

Additional excipients were screed using DLS at the same 333:1 polymer:mRNA ratio, substituting half of the polymer with various excipients (FIG. 14). PVP-PVA 1:1 with no additional excipient outperformed all of the tested combinations, which further evidences the unexpected nature of the composition and its ability to form a matrix that can support mRNA-LNP re-dispersion and subsequent bioactivity.

In Vivo Shelf Life Study with Microneedle Patches Containing mRNA-LNPs

Using the methods previously described, microneedle patches containing approximately 1 μg of SARS-CoV-2 receptor binding domain (RBD) mRNA were fabricated. These patches were stored for either 1 or 3 months at 4° C. or room temperature (RT). All patches were stored in sealed petri dishes with desiccant to remove moisture. SARS-CoV-2 mRNA-LNPs in liquid suspension were stored in identical conditions to provide a control. All mice used for this study were primed with a fresh 10 μg dose of SARS-CoV-2 RBD mRNA in LNPs in liquid suspension via IM injection. Four weeks after the prime dose the booster dose was delivered via either stored microneedle patches administered intradermally or stored suspension administered intramuscularly (IM).

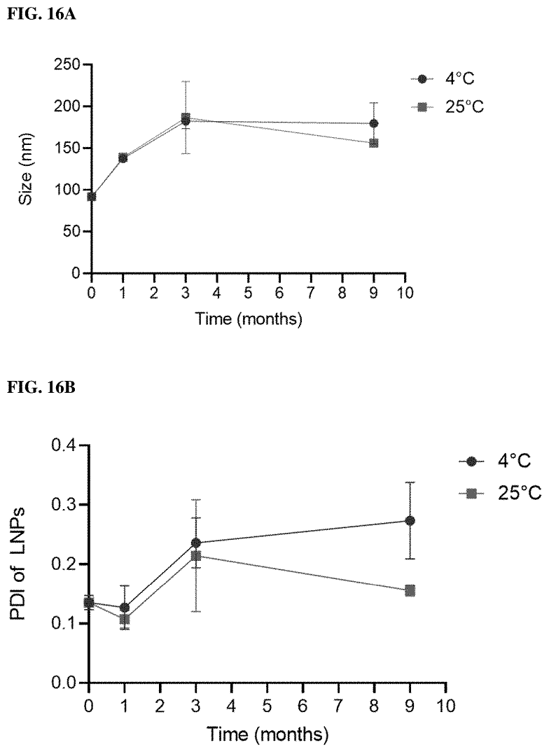

Stored microneedle patches and stored liquid suspension perform comparably, providing a significant boost in anti-SARS-CoV-2 RBD IgG antibody responses, as measured in relative light units by an electrochemiluminescent binding assay (FIGS. 15A & 15B). This provides further evidence for the immunogenicity and stability of microneedle patches containing mRNA-LNPs.

The mRNA-LNP liquid suspension was further characterized over time. The variance and PDI of LNP size go up over time, showing how the qualities of a liquid vaccine can change. Though it remains immunogenic, uncontrolled aggregation of the liquid mRNA-LNP suspension could affect the overall consistency and safety of the liquid suspension vaccine when administered IM. It is expected that LNPs in microneedle patches will remain static since they are immobilized by the surrounding PVP-PVA polymer matrix. This effect will provide a consistent product at any time after fabrication, which is a significant advantage.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition, comprising:
i) a polymer or a copolymer and a lipid nanoparticle, wherein the w/w ratio of the polymer or the copolymer to the lipid nanoparticle is at least 100:1; or
ii) a polymer or a copolymer and a biomolecule, wherein the w/w ratio of the polymer or the copolymer to the biomolecule is at least 1000:1.

2. The composition of claim 1, wherein the composition comprises a polymer or a copolymer and a lipid nanoparticle, wherein the w/w ratio of the polymer or the copolymer to the lipid nanoparticle is at least 100:1.

3. (canceled)

4. (canceled)

5. The composition of claim 1, wherein the lipid nanoparticle comprises an ionizable lipid, cholesterol, a polyethyleneglycol lipid, a phospholipid, or a combination thereof.

6. The composition of claim 1, wherein the lipid nanoparticle comprises 1-octylnonyl 8-[(2-hydroxyethyl)[8-(nonyloxy)-8-oxooctyl]amino]octanoate (i.e., lipid 5, CAS Ref. No.: 2089251-33-0) or 3,6-bis(4-(bis(2-hydroxydodecyl)amino)butyl)piperazine-2,5-dione (i.e., cKK-E12).

7-9. (canceled)

10. The composition of claim 1, wherein the diameter of the lipid nanoparticle is about 50 nm, about 75 nm, about 100 nm, about 125 nm, about 150 nm, about 175 nm, about 200 nm, about 225 nm, about 250, about 275 nm, or about 300 nm.

11. (canceled)

12. (canceled)

13. The composition of claim 1, further comprising a biomolecule.

14-17. (canceled)

18. The composition of claim 1, wherein the composition comprises a polymer and the polymer is polyvinylpyrrolidone or polyvinylalcohol.

19-21. (canceled)

22. The composition of claim 1, wherein the composition comprises a copolymer and the copolymer comprises a plurality of repeat units of vinylalcohol; a plurality of repeat units of vinylpyrrolidinone; and the copolymer is a block copolymer or a random copolymer.

23-26. (canceled)

27. The composition of claim 1, wherein the copolymer comprises a plurality of repeat units of vinylalcohol; a plurality of repeat units of sucrose; and the copolymer is a block copolymer or a random copolymer.

28-30. (canceled)

31. The composition of claim 1, wherein the copolymer comprises a plurality of repeat units of vinylalcohol, a plurality of repeat units of polyvinylpyrrolidone, a plurality of repeat units of sucrose; and the copolymer is a block copolymer or a random copolymer.

32-34. (canceled)

35. The composition of claim 1, wherein the copolymer is a block copolymer.

36. The composition of claim 1, wherein the copolymer is a random copolymer.

37. The composition of claim 1, wherein the polymer comprises 50-500 repeat units.

38-41. (canceled)

42. The composition of claim 1, wherein the copolymer comprises 250-1,500 repeat units.

43-47. (canceled)

48. The composition of claim 13, wherein the biomolecule is an RNA.

49. (canceled)

50. The composition of claim 13, wherein the concentration of the biomolecule is about 50 μg/mL, about 100 μg/mL, about 150 μg/mL, about 200 μg/mL, about 250 μg/mL, about 300 μg/mL, about 350 μg/mL, about 400 μg/mL, about 450 μg/mL, about 500 μg/mL, about 550 μg/mL, about 600 μg/mL, about 650 μg/mL about 700 μg/mL, about 750 μg/mL, about 800 μg/mL, about 850 μg/mL, about 900 μg/mL, about 950 μg/mL or about 1,000 μg/mL.

51-54. (canceled)

55. A microneedle array, comprising the composition of claim 1.

56-63. (canceled)

64. A polymeric device comprising a polymeric shell and at least one discrete region comprising the composition of claim 1, optionally in combination with a stabilizing excipient, wherein the shell and discrete regions are formed from successive layers of polymeric particles bonded together by solvent and/or temperature by three dimensional printing or micromolding.

65-88. (canceled)

89. A microdevice having dimensions of less than one centimeter comprising a micromolded fillable polymeric shell comprising a non-photoactivatable biocompatible polymer having a complex channel or core therein; wherein the channel or core contains the composition of claim 1.

90-97. (canceled)

98. A method of delivery a therapy, comprising contacting a subject in need thereof with an effective amount of the composition of claim 1.

99. (canceled)

100. (canceled)
